(19) United States
(12) Patent Application Publication
(10) Pub. No.: US 2023/0111948 A1
(43) Pub. Date: Apr. 13, 2023

(54) METHODS FOR TREATING CANCER BY ADJUSTING SELECTED METABOLITES

(71) Applicant: Gus Lawrence, Portland, OR (US)

(72) Inventor: Gus Lawrence, Portland, OR (US)

(21) Appl. No.: 17/498,188

(22) Filed: Oct. 11, 2021

(51) Int. Cl. A01N 1/02

(52) U.S. Cl. CPC A01N 1/021

(57) ABSTRACT

Cancer treatment methods that include adjusting the concentration of selected metabolites in a blood supply to yield a perfusate with metabolite concentrations that promote terminal cellular differentiation in a tumor and delivering the perfusate to an artery of a patient. In some examples, the methods include adjusting the concentration of glycerol in a blood supply to yield a perfusate with reduced osmotic pressure, adjusting the concentration of ascorbic acid in the perfusate to maintain a selected redox potential, adjusting the concentration of glutamine in the perfusate to downregulate histone deacetylases, adjusting the concentration of butyrate in the perfusate to signal starvation, and delivering the perfusate to an artery of a patient, wherein the concentrations of glycerol, ascorbic acid, glutamine, and butyrate are selected to promote terminal cellular differentiation in a tumor.

### BACKGROUND

The present disclosure relates generally to methods of treating cancer. In particular, methods of treating cancer by adjusting selected metabolites are described.

The history of life and metabolism is recorded in the order of biosynthetic pathways and the coenzymes of metabolism. Like the history of metabolism, the history of human development is recapitulated during embryogenesis at the level of gene expression. Thus, early developmental stages of the embryo represent the structure of a primordial life form just evolving multicellularity.

Fick's Law predicts the metabolic characteristics of interior and exterior cells. These characteristics will drive the differentiation and dedifferentiation of cells. Thus, metabolic conditions consistent with “inner” cells will inhibit differentiation and metabolic conditions consistent with “outer” cells will promote differentiation. Accordingly, these conditions drive differentiation in the embryo.

Hyperosmotic conditions inhibit cell differentiation and promote the expression of early markers of the inner cell mass. Conversely, hyposmotic conditions downregulate markers of the inner cell mass.

Hypoosmotic stress downregulates the stem cell marker Oct4 and upregulates E-cadherin. E-cadherin is one of the first markers of differentiation in the embryo and a marker of the mesenchymal to epithelial transition.

Oxygen gradients control differentiation patterns in the early embryo, similar to osmotic forces. Hypoxia is critical for the stem cell niche and hypoxia induces dedifferentiation and promotes cancer stem cells. Hyperbaric oxygen increases cancer survival in conjunction with chemotherapy. Similarly, acidosis promotes cellular de-differentiation and an alkaline diet increases therapeutic response to chemotherapy.

Known cancer treatment methods are not entirely satisfactory for the range of applications in which they are employed. For example, existing cancer treatment methods, such as neoadjuvant therapies, often cause unintended damage to tissue and organs in an effort to eradicate cancer cells.

Neoadjuvant therapies sensitize cancer to subsequent treatments and can cause conventional cancer treatments to become less effective. Cancer treatments and neoadjuvant therapies are almost exclusively cytolytic. These conventional therapies are expensive, produce systemic side effects, and can generate treatment resistance.

Cancer is not simply unchecked proliferation or inadequate immune surveillance. Cancer is also a failure of the cancer cell to differentiate. Conventional cancer treatment methods do not adequately induce cancer cells to differentiate. Further, conventional cancer treatment methods do not adequately utilize natural metabolic pathways to encourage cancer cell differentiation.

Thus, there exists a need for cancer treatment methods that improve upon and advance known cancer treatment methods. Examples of new and useful cancer treatment methods relevant to the needs existing in the field are discussed below.

### SUMMARY

The present disclosure is directed to methods of treating cancer that include adjusting the concentration of selected metabolites in a blood supply to yield a perfusate with metabolite concentrations that promote terminal cellular differentiation in a tumor and delivering the perfusate to an artery of a patient. In some examples, the methods include adjusting the concentration of glycerol in a blood supply to yield a perfusate with reduced osmotic pressure, adjusting the concentration of ascorbic acid in the perfusate to maintain a selected redox potential, adjusting the concentration of glutamine in the perfusate to downregulate histone deacetylases, adjusting the concentration of butyrate in the perfusate to signal starvation, and delivering the perfusate to an artery of a patient, wherein the concentrations of glycerol, ascorbic acid, glutamine, and butyrate are selected to promote terminal cellular differentiation in a tumor.

### BRIEF DESCRIPTION OF THE DRAWINGS

FIG. 1 is a flow diagram of a first example of a cancer treatment method.

FIG. 2 is a flow diagram showing sub-steps of a metabolite concentration adjustment step of the cancer treatment method shown in FIG. 1.

FIG. 3 is a flow diagram of sub-steps of an alternative metabolite concentration adjustment step.

### DETAILED DESCRIPTION

The disclosed cancer treatment methods will become better understood through review of the following detailed description in conjunction with the figures. The detailed description and figures provide merely examples of the various inventions described herein. Those skilled in the art will understand that the disclosed examples may be varied, modified, and altered without departing from the scope of the inventions described herein. Many variations are contemplated for different applications and design considerations; however, for the sake of brevity, each and every contemplated variation is not individually described in the following detailed description.

Throughout the following detailed description, examples of various cancer treatment methods are provided. Related features in the examples may be identical, similar, or dissimilar in different examples. For the sake of brevity, related features will not be redundantly explained in each example. Instead, the use of related feature names will cue the reader that the feature with a related feature name may be similar to the related feature in an example explained previously. Features specific to a given example will be described in that particular example. The reader should understand that a given feature need not be the same or similar to the specific portrayal of a related feature in any given figure or example.

### Definitions

The following definitions apply herein, unless otherwise indicated.

“Substantially” means to be more-or-less conforming to the particular dimension, range, shape, concept, or other aspect modified by the term, such that a feature or component need not conform exactly. For example, a “substantially cylindrical” object means that the object resembles a cylinder, but may have one or more deviations from a true cylinder.

“Comprising,” “including.” and “having” (and conjugations thereof are used interchangeably to mean including but not necessarily limited to, and are open-ended terms not intended to exclude additional elements or method steps not expressly recited.

Terms such as “first”, “second”, and “third” are used to distinguish or identify various members of a group, or the like, and are not intended to denote a serial, chronological, or numerical limitation.

“Coupled” means connected, either permanently or releasably, whether directly or indirectly through intervening components.

Methods for Treating Cancer by Adjusting Selected Metabolites

With reference to the figures, methods for treating cancer by adjusting selected metabolite concentrations will now be described. The methods discussed herein function to treat cancer. In particular, the methods functions to treat cancer by utilizing specific metabolic pathways.

The reader will appreciate from the figures and description below that the presently disclosed cancer treatment methods address many of the shortcomings of conventional cancer treatment methods. For example, the novel methods below reduce or completely avoid unintended damage to tissue and organs when working to stop the growth of cancer cells. Further, the novel cancer treatment methods described herein utilize natural metabolic pathways to harness the body's inherent ability to fight cancer.

Metabolic preconditioning using the methods described below has the potential to replace neoadjuvant therapy. Metabolic preconditioning according to the methods descried herein is low cost, safe, and utilizes a clear and definitive mechanism with a high likelihood of success.

Technology has advanced enough that isolating a tumor's blood supply and supplying the tumor with a nutrient profile that promotes differentiation according to the methods discussed in this document is possible. The cancer treatment methods described herein can shrink tumors that cannot be surgically resected or are in vital organs like the brain or pancreas. Metabolic preconditioning using the methods below can dramatically reduce treatment costs and side effects. Additionally, the novel methods discussed in this document can make immunotherapy and chemotherapy more effective without off-target effects.

Cancer Treatment Method Embodiment One

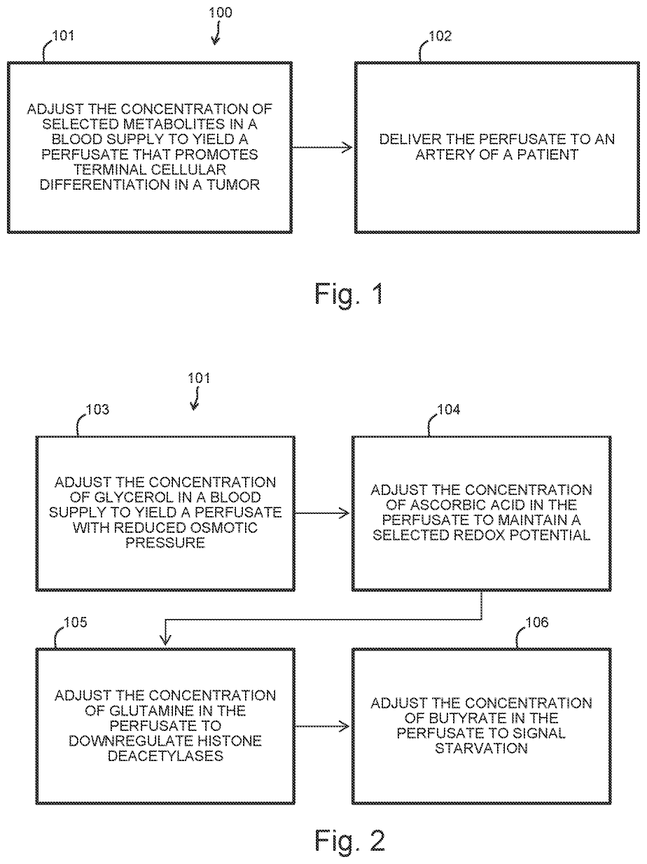

With reference to FIGS. 1 and 2, a first example of a cancer treatment method, cancer treatment method 100, will now be described. As shown in FIG. 1, cancer treatment method 100 includes the steps of adjusting the concentration of selected metabolites in a blood supply to yield a perfusate at step 101 and delivering the perfusate to an artery of a patient at step 102. In some examples, the cancer treatment method does not include one or more aspects of cancer treatment method 100. In other examples, the cancer treatment method includes additional or alternative steps.

The steps of cancer treatment method 100 will be described in more detail in the sections below.

Adjusting Selected Metabolite Concentrations

As can be seen in FIG. 1, adjusting the concentration of selected metabolites in a blood supply at step 101 yields a perfusate that is delivered to a patient in step 102. The concentrations of the selected metabolites are adjusted to promote terminal cellular differentiation in a tumor. Promoting terminal cellular differentiation in the tumor functions to force the tumor to exit the cell cycle. Forcing the tumor to exit the cell cycle dramatically improves cancer cure rates, including in leukemia and endometrial cancer.

In some examples, the concentrations of selected metabolites in the blood supply are adjusted to reduce the osmotic pressure of the perfusate. Additionally or alternatively, the concentration of selected metabolites in the blood supply may be adjusted to maintain a selected redox potential of the perfusate. The selected redox potential may correspond to a selected concentration of intra-cellular glutathione that promotes apoptosis in a tumor.

In certain examples, the concentrations of selected metabolites in the blood supply are adjusted to signal starvation. Additionally or alternatively, the concentration of selected metabolites in the blood supply may be adjusted to regulate expression of the Yamanaka stem cell factors in the perfusate. Further, the concentrations of selected metabolites in the blood supply may be adjusted to restore a patient's compromised immune response to a tumor to a baseline immune response. Another basis for adjusting the concentration of selected metabolites in the blood supply is restoring extracellular oxidation reduction potential in an environment proximate a tumor to within a baseline range of an extracellular oxidation reduction potential.

With reference to FIG. 2, the reader can see the steps included in adjusting the concentration of selected metabolites in a blood supply at step 101. In the example shown in FIG. 2, adjusting the concentration of selected metabolites in a blood supply at step 101 includes adjusting the concentration of glycerol in a blood supply to yield a perfusate with reduced osmotic pressure at step 103. Step 101 further includes adjusting the concentration of ascorbic acid in the perfusate to maintain a selected redox potential at step 104. Step 101 continues with adjusting the concentration of glutamine in the perfusate to downregulate histone deacetylases at step 105. A further sub-step of step 101 is adjusting the concentration of butyrate in the perfusate to signal starvation at step 106.

The concentrations of glycerol, ascorbic acid, glutamine, and butyrate are adjusted in step 101 to selected metabolite concentrations. The metabolite concentrations resulting from the step 101 adjustments are selected to promote terminal cellular differentiation in a tumor.

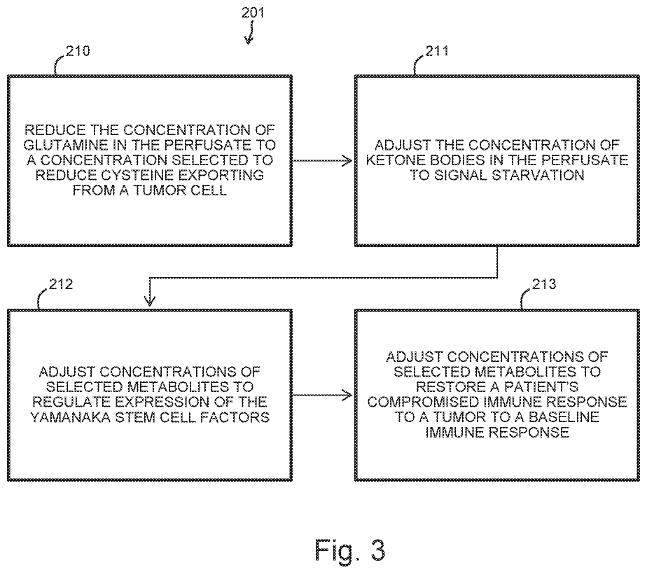

Each sub-step of adjusting the concentration of selected metabolites in a blood supply at step 101 will be described in the sections below. The reader should understand, however, that some examples of adjusting the concentration of selected metabolites in a blood supply include fewer, additional, or different steps than depicted in FIG. 2. For example, FIG. 3 depicts a variation of adjusting the concentration of selected metabolites in a blood supply with different sub-steps, steps 210-213, than depicted in FIG. 2.

Adjusting the Concentration of Glycerol

Adjusting the concentration of glycerol at step 103 functions to yield a perfusate with reduced osmotic pressure. Reducing osmotic pressure controls the stem cell factor Oct4. Osmotic conditions can also be used to control endocytosis.

Cancer cells upregulate endocytosis to increase uptake of amino acids and membranes and to bias recycling of integrins and signaling receptors. Endocytosis is the rate limiting step in cadherin downregulation and is crucial for the EMT and cancer invasiveness. Endosomes integrate Tor-Akt and MAP kinase signaling, and they form the scaffold for the enzymes of nucleotide synthesis.

Hypoosmotic conditions inhibit endocytosis and promote exocytosis. Hyposmotic swelling can be achieved by exposure to membrane permeant osmolytes followed by passive transport of water. Glycerol is a nontoxic, metabolizable, membrane permeant osmolyte.

Glycerol inhibits endocytosis and induces exocytosis causing cell swelling. Unlike other membrane permeant osmolytes such as urea, glycerol induces exocytosis for long periods because it is phosphorylated and enters central metabolism.

Glycerol accumulation opposes glucose import preventing the rapid metabolism required for cell division. Respiration on glycerol and AMPK signaling physically disrupts an endosomal/mitochondrial junction necessary for rapid channeling of substrates into nucleotide synthesis. Because glycerol is a membrane permeant osmolyte it doesn't induce osmoadaptation. Thus, multiple cycles of glycerol perfusion, metabolic trapping, water influx, and cell swelling can be performed.

In the present example, step 103 includes increasing the concentration of glycerol in the perfusate. For example, the concentration of glycerol in the perfusate may be increased to 50 mM. In some example, the concentration range of glycerol in the perfusate is selected to be between 45 and 55 mM. The concentration of glycerol may be adjusted to any concentration effective to reduce osmotic pressure and control endocytosis

Adjusting the Concentration of Ascorbic Acid

Adjusting the concentration of ascorbic acid in the perfusate at step 104 functions to maintain a selected redox potential. Hyperoxia opposes transcription of stemness factors. Hyperbaric oxygen promotes better outcomes in conventional cancer treatments.

Changes in the extracellular oxidation reduction potential predict and control the immune response. The tumor microenvironment and redox state signaling predicts immune response and survival time. Hypoxia impairs beneficial macrophage polarization and determines NK cell differentiation.

Hyperbaric oxygen increases T-cell infiltration to the tumor. Regulatory T-cells use extracellular redox remodeling to suppress the immune response. Glutathione primes T-cells for inflammation. Redox signaling is key to orchestrate the immune response, and engineering these signals in the tumor environment will increase the immune response to the tumor.

The redox state can also be used to manipulate other cellular pathways. Pentose phosphate pathway (PPP) provides nucleotides for rapidly dividing cells. The PPP is regulated by dehydroascorbate. Ascorbate kills cancer stem cells, promotes expression of the cell cycle genes that inhibit cell cycle progression, and inhibits glucose consumption while stimulating lactate transport.

Using specialized perfusates to adjust the oxidation reduction potential can promote cell differentiation, impair nucleotide synthesis, and restore the cells' ability to commit apoptosis. Restoring the proper redox signaling allows the immune system to activate in the tumor microenvironment.

Intracellular redox state determines a cell's decision to self-renew, differentiate, or commit apoptosis. The intracellular reduction potential is sensed and indicated by molecules like glutathione and ascorbic acid, which are in equilibrium. Thus, adjusting the concentration of ascorbic acid enables controlling the intracellular redox potential, and, in turn, a cancer cell's tendencies to self-renew, differentiate, or commit apoptosis.

In some examples, a selected redox potential of the perfusate is maintained by adjusting the concentration of ascorbic acid (or ascorbate) in the perfusate. The selected redox potential corresponds to a selected concentration of intra-cellular glutathione that promotes apoptosis in a tumor. In some examples, the concentration of ascorbic acid is increased to between 1 and 2 mM to maintain the selected redox potential. Higher or lower ascorbic acid concentrations may be used to maintain a desired redox potential.

Adjusting the Concentration of Glutamine

Adjusting the concentration of glutamine in the perfusate at step 105 has multiple functions. One function is to downregulate histone deacetylases. A further function is to maintain expression of the stem cell transcription factor Oct4. Another function is to reduce impairments to tumor cells differentiating.

Glutamine is the major anaplerotic substrate and the initial substrate in the nucleotide synthesis that maintains cancer stem cells. Glutamine supplies aKG, which is the substrate for DNA demethylases. DNA demethylases directly impairs differentiation.

Glutamine also impairs differentiation by downregulating histone deacetylases (HDACs). Further, glutamine maintains expression of the stem cell transcription factor Oct4. Limiting the utilization of glutamine makes cells glucose dependent.

Glutamine modulates the cell redox state. Changing the redox potential promotes nucleotide synthesis. Glutamine withdrawal depletes the endogenous antioxidant glutathione (GSH) and OCT4 is subsequently inactivated via cysteine oxidation and degraded, which leads to cell differentiation.

Glutamine concentration is up to approximately 800 mM in normal conditions. In withdrawal conditions, glutamine concentration approaches approximately 200 mM. The present methods include decreasing glutamine concentrations in the perfusate to values near 200 mM. In some examples, the glutamine concentrations are decreased to between 200 mM and 260 mM. In other examples, the glutamine concentration is decreased to between 150 mM and 350 mM.

Adjusting the Concentration of Butyrate

Adjusting the concentration of butyrate in the perfusate at step 106 functions to signal starvation. Lipid metabolism heavily influences cell differentiation and expression of stem cell markers like Myc.

Myc transcription is repressed by the Myc promoter-binding protein-1 (MBP-1). MBP-1 is related to the glycolytic enzyme alpha-enolase and is regulated by hypoxia.

A ketogenic diet potentiates chemotherapies and radiation treatments and increases cancer survival. Ketone bodies like butyrate are endogenous histone deacetylase inhibitors (HDACi). Butyrate inhibits the histone deacetylases that control sternness to promote tissue differentiation.

HDACI promote differentiation, in part, by the selective depletion of Bromo Domain (BET) proteins. Depleting BET proteins directly targets transcription of Myc.

Butyrate acts in the mitochondrial apoptotic pathway. Butyrate is more effective as an HDACi if lipid metabolism is active. Supplements that accelerate lipid consumption, including alpha lipoic acid, acetylcarnitine CoQ10, or pantethine, all sensitize cancer to treatment and improve survival. Thus, as part of this metabolic therapy, to make butyrate effective as an HDAC, patients would take these supplements in conjunction with metabolite perfusion to increase the effectiveness of the butyrate therapy.

Butyrate broadly downregulates transcription, consistent with downregulation of Myc. Butyrate levels correlate with therapeutic response in anti CTLA4 and anti PD-L1 immunotherapy in melanoma. The antitumor response relies on toll-like receptor 4 (TLR)-4 signaling.

In some examples, adjusting the concentration of butyrate in the perfusate to signal starvation includes increasing the concentration of butyrate in the perfusate. In one specific example, butyrate is increased to a concentration of 6 to 8 mM in the perfusate. The concentration of butyrate may be increased to smaller or larger concentrations effective to signal starvation in cancer cells.

Delivering the Perfusate

Delivering the perfusate to an artery of a patient at step 102 functions to enable the perfusate established in step 101 to be distributed throughout the patient's body by the patient's cardiovascular system. When the perfusate is distributed throughout the patient's body, the metabolites in the perfusate at selected concentrations can interact with cells and drive beneficial terminal differentiation of tumor cells.

Delivering the perfusate at step 102 may be accomplished by any currently known or later developed means. Suitable means include hemodialysis machines, extracorporeal membrane oxygenation (ECMO) techniques and machines, and simple injections.

### Additional Embodiments

With reference to the figures not yet discussed, the discussion will now focus on additional cancer treatment method embodiments. The additional embodiments include many similar or identical features to cancer treatment method 100. Thus, for the sake of brevity, each aspect of the additional embodiments below will not be redundantly explained. Rather, key distinctions between the additional embodiments and cancer treatment method 100 will be described in detail and the reader should reference the discussion above for features substantially similar between the different cancer treatment method examples.

### Second Embodiment

Turning attention to FIG. 3, a second example of a cancer treatment method will now be described. The second example of a cancer treatment method includes the steps of adjusting the concentration of selected metabolites in a blood supply to yield a perfusate at step 201 and delivering the perfusate to an artery of a patient.

As can be seen in FIG. 3, adjusting the concentration of selected metabolites in a blood supply to yield a perfusate at step 201 includes different steps than corresponding step 101 in FIG. 2. With reference to FIG. 3, step 201 includes the sub-steps of reducing the concentration of glutamine in the perfusate at step 210, adjusting the concentration of ketone bodies in the perfusate at step 211, adjusting concentrations of selected metabolites to regulate expression of the Yamanaka stem cell factors at step 212, and adjusting concentrations of selected metabolites to restore a patient's baseline immune response to a tumor at step 213. The sub-steps of step 201 will be discussed in the sections below.

Adjusting the Concentration of Glutamine

Step 210 depicted in FIG. 3 involves reducing the concentration of glutamine in the perfusate to a concentration selected to reduce cysteine exporting from a tumor cell. Redox potential, cysteine, and glutamine converge upon the metabolic decisional circuitry via glutathione.

Glutathione is synthesized from glutamate and cysteine and its synthesis is dependent upon the cellular redox state. High GSH (glutathione) synthase activity maintains stem cells. Apoptosis is regulated by the intra-cellular levels of GSH and dependent upon the GSH-related redox pattern of the cell.

The importance of cysteine in stemness and failure to differentiate is evidenced by the cysteine/glutamate exchange transporter (xCT). xCT is essential for KRAS-mediated transformation. This transporter promotes stem cell properties and is implicated in very aggressive cancers, such as glioblastoma and triple negative breast cancer.

xCT activates the pentose phosphate pathway (PPP) via oxidative stress from cysteine. Import of cysteine and conversion to cysteine allows synthesis of glutathione.

Export of cysteine leads to a more reducing oxidation potential in the extracellular environment. This change in extracellular redox state helps defeat the immune response to the cancer.

Adjusting the Concentration of Ketone Bodies

Adjusting the concentration of ketone bodies in the perfusate at step 211 functions to signal starvation. As discussed above with regard to the ketone body Butyrate, ketone bodies are endogenous histone deacetylase inhibitors (HDACi). Ketone bodies inhibit the histone deacetylases that control stemness to promote tissue differentiation.

Ketone bodies broadly downregulate transcription similarly to downregulating Myc. Ketone body levels correlate with therapeutic response in anti CTLA4 and anti PD-L1 immunotherapy in melanoma. The antitumor response to ketone body levels relies on toll-like receptor 4 (TLR)-4 signaling.

In some examples, adjusting the concentration of ketone bodies in the perfusate to signal starvation includes increasing the concentration of ketone bodies in the perfusate. In one specific example, ketone bodies are increased to a concentration of 6 to 8 mM in the perfusate. The concentration of ketone bodies may be increased to smaller or larger concentrations effective to signal starvation in cancer cells.

Regulating Expression of Yamanaka Stem Cell Factors

Adjusting concentrations of selected metabolites to regulate expression of the Yamanaka stem cell factors at step 212 functions to induce pluripotency or stemness. Four Yamanaka transcription factors are sufficient to induce stemness.

Expression of the four Yamanaka transcription factors is a marker of dedifferentiation and is a negative prognostic sign in cancer. Downregulation of these markers is associated with the process of differentiation. The primordial determinants of cellular differentiation also control the stem cell factors.

OCT4 is a redox-based sensor of glutamine metabolism. Hypoosmotic signals lead to its phosphorylation and degradation. Sox2 is downregulated by hyperoxia. Hypoxia allows MYC transcription. MYC amplifies RNA expression, mitigates cellular acidity, and enhances glutamine metabolism.

Restoring a Patient's Compromised Immune Response

Restoring a patient's compromised immune response to a tumor at step 213 functions to reestablish a baseline immune response for the patient. Adjusting metabolic concentrations of selected metabolites in the perfusate enables addressing conditions where redox remodeling is disabled in a patient's immune response. Restoring redox remodeling signals enables the patient's immune response to the tumor to be restored to baseline levels rather than compromised and ineffectual levels.

Restoring a patient's compromised immune response may be accomplished by adjusting one or more concentrations of metabolites in the perfusate. The restoring concentrations may correspond to one or more of the metabolite concentration ranges above. For example, the concentration of ascorbate in the perfusate may be adjusted to 1 to 2 mM.

### REFERENCES INCORPORATED BY REFERENCE

References relevant to cancer treatment methods include the following references, the complete disclosures of which are incorporated herein by reference for all purposes:
Kwan, J. & Pua, U. Review of intra-arterial therapies for colorectal cancer liver metastasis. Cancers 13, 1-27 (2021).
Pusuluri, A. et al Role of synergy and immunostimulation in design of chemotherapy combinations: An analysis of doxorubicin and camptothecin. Bioengineering & Translational Medicine 4, (2019).
Enane, F. O., Saunthararajah, Y. & Korc, M. Differentiation therapy and the mechanisms that terminate cancer cell proliferation without harming normal cells. Cell Death Dis. 9, (2018).
Buteyn, N. J. et al Anti-leukemic effects of all-trans retinoic acid in combination with Daratumumab in acute myeloid leukemia. Int. Immunol. 30, 375-383 (2018).
Kim, J. J. & Chapman-Davis, E. Role of progesterone in endometrial cancer. Semin. Reprod. Med. 28, 81-90 (2010).
Lee, J. V. et al. Acetyl-CoA promotes glioblastoma cell adhesion and migration through Ca2+-NFAT signaling. Genes Dev. 32, 497-511 (2018).
Montgomery, D. C., Sorum, A. W., Guasch, L., Nicklaus, M. C. & Meier, J. L. Metabolic Regulation of Histone Acetyltransferases by Endogenous Acyl-CoA Cofactors. Chem. Biol. 22, 1030-1039 (2015).
Shen, Y., Wei, W. & Zhou, D.-X. Histone Acetylation Enzymes Coordinate Metabolism and Gene Expression. Trends Plant Sci. 20, 614-621 (2015).
Lasko, L. M. et al Discovery of a selective catalytic p300/CBP inhibitor that targets lineage-specific tumours. Nature 550, 128-132 (2017).
Wang, Y. C., Peterson, S. E. & Loring, J. F. Protein post-translational modifications and regulation of pluripotency in human stem cells. Cell Res. 24, 143-160 (2014).
Dang, L. et al. Cancer-associated IDH1 mutations produce 2-hydroxyglutarate. Nature 462, 739-44 (2009).
Reitman, Z. J. & Yan, H. Isocitrate dehydrogenase 1 and 2 mutations in cancer: alterations at a crossroads of cellular metabolism. J. Natl. Cancer Inst. 102, 932-41 (2010).
Feuerecker, B. et al Lipoic acid inhibits cell proliferation of tumor cells in vitro and in vivo. Cancer Biol. Ther. 13, 1425-1435 (2012).
Baci, D. et al Acetyl-L-Camitine downregulates invasion (CXCR4/CXCL12, MMP-9) and angiogenesis (VEGF, CXCL8) pathways in prostate cancer cells: rationale for prevention and interception strategies. J. Exp. Clin. Cancer Res. 38, 1-17 (2019).
Frontiñán-Rubio, J. et al Regulation of the oxidative balance with coenzyme Q10 sensitizes human glioblastoma cells to radiation and temozolomide. Radiother. Oncol. 128, 236-244 (2018).
Penet, M. F. et al Effect of Pantethine on Ovarian Tumor Progression and Choline Metabolism. Front. Oncol. 6, 1-9 (2016).
Gröber, U., Holzhauer, P., Kisters, K., Holick, M. F. & Adamietz, I. A. Micronutrients in oncological intervention. Nutrients 8, 1-30 (2016).
Kraft, M. et al L-Carnitine-supplementation in advanced pancreatic cancer (CARPAN)—a randomized multicentre trial. 4-9 (2012).
Park, S., Ahn, S., Shin, Y., Yang, Y. & Yeom, C. 11. Vitamin C in cancer: A metabolomics perspective. Front. Physiol. 9, 1-9 (2018).
Nauman, G., Gray, J. C., Parkinson, R., Levine, M. & Paller, C. J. Systematic review of intravenous ascorbate in cancer clinical trials. Antioxidants 7, 1-22 (2018).
Stephenson, C. M., Levin, R. D., Spector, T. & Lis, C. G. Phase i clinical trial to evaluate the safety, tolerability, and pharmacokinetics of high-dose intravenous ascorbic acid in patients with advanced cancer. Cancer Chemother. Pharmacol. 72, 139-146 (2013).
Gao, X. et al. Dietary methionine influences therapy in mouse cancer models and alters human metabolism. Nature 572, 397-401 (2019).
White, H. B. Coenzymes as fossils of an earlier metabolic state. J. Mol. Evol. 7, 101-104 (1976).
Clune, J., Pennock, R. T., Ofria, C. & Lenski, R. E. Ontogeny Tends to Recapitulate Phylogeny in Digital Organisms. Am. Nat. 180, E5-4-E63 (2012).
Kalinka, A. T. et al. Gene expression divergence recapitulates the developmental hourglass model. Nature 468, 811-816 (2010).
Madonna, R., Geng, Y. J., Shelat, H., Ferdinandy, P. & De Caterina, R. High glucose-induced hyperosmolarity impacts proliferation, cytoskeleton remodeling and migration of human induced pluripotent stem cells via aquaporin-1. Biochem. Biophys. Acta—Mol. Basis Dis. 1842, 2266-2275 (2014).
TANAKA, T., TAKEI, Y., ZAIMA, N., MORIYAMA, T. & YAMANOUCHI, D. Hyperglycemia Suppresses RANKL-Induced Osteoclast Differentiation through LXR&amnp;beta; Expression in RAW264.7 Cells. J. Nutr. Sci. Vitaminol. (Tokyo). 63, 28-34 (2017).
Then, C. et al. Hypertonic stress promotes the upregulation and phosphorylation of zonula occludens 1. Nephron—Physiol. 119, p11-p21 (2011).
Wang, H. et al. transformation in the mouse. 318, 112-125 (2009).
Sheth, B. et al. Tight junction protein ZO-2 expression and relative function of ZO-1 and ZO-2 during mouse blastocyst formation. Exp. Cell Res. 314, 3356-3368 (2008).
Chin, S., Bogliolo, S., Ehrenfeld, J. & Ciapa, B. Activation of extracellular signal-regulated kinase ERK after hypo-osmotic stress in renal epithelial A6 cells. Biochim. Biophys. Acta—Biomembr. 1664, 224-229 (2004).
Fouchs, A. et al Activation of the MAPKs ERK1/2 by cell swelling in turbot hepatocytes. Biol. cell 102, 447-456 (2010).
Brumbaugh, J. et al Phosphorylation regulates human OCT4. Proc. Natl. Acad. Sci. 109, 7162-7168 (2012).
Kippenberger, S. et al. Hypotonic stress induces E-cadherin expression in cultured human keratinocytes. FEBS Lett. 579, 207-214 (2005).
Mulas, C. et al Oct4 regulates the embryonic axis and coordinates exit from pluripotency and gemi layer specification in the mouse embryo. Development 145, (2018).
Fathollahipour, S., Patil, P. S. & Leipzig, N. D. Oxygen regulation in development: Lessons from embryogenesis towards tissue engineering. Cells Tissues Organs 205, 350-371 (2019).
Mohyeldin, A., Garzón-Muvdi, T. & Quinones-Hinojosa, A. Oxygen in stem cell biology: A critical component of the stem cell niche. Cell Stem Cell 7, 150-161 (2010).
Axelson, H., Fredlund, E., Ovenberger, M., Landberg, G. & Pählman, S. Hypoxia-induced dedifferentiation of tumor cells—A mechanism behind heterogeneity and aggressiveness of solid tumors. Semin. Cell Dev. Biol. 16, 554-563 (2005).
Yamada, Y., Haga, H. & Yamada, Y. Concise Review: Dedifferentiation Meets Cancer Development: Proof of Concept for Epigenetic Cancer. Stem Cells Transl. Med. 3, 1182-1187 (2014).
Wang, P., Wan, W., Xiong, S.-L., Feng, H. & Wu, N. Cancer stem-like cells can be induced through dedifferentiation under hypoxic conditions in glioma, hepatoma and lung cancer. Cell Death Discov. 3, 16105 (2017).
Bennett, M. H., Feldmeier, J., Smee, R. & Milross, C. Hyperbaric oxygenation for tumour sensitisation to radiotherapy. Diving Hyperb. Med. 48, 116-117 (2018).
Agathocleous, M. & Harris, W. A. Metabolism in physiological cell proliferation and differentiation. Trends Cell Biol. 23, 484-492 (2013).
Intlekofer, A. M. et al Hypoxia Induces Production of L-2-Hydroxyglutarate. Cell Metab. 22, 304-311 (2015).
Hamaguchi, R., Okamoto, T., Sato, M. & Wada, H. Effects of an Alkaline Diet on EGFR-TKI Therapy in EGFR Mutation-positive NSCLC. Anticancer Res. 37, 5141-5145 (2017).
Nagata, S. et al Efficient reprogramming of human and mouse primary extra-embryonic cells to pluripotent stem cells. Genes Cells 14, 1395-404 (2009).
Marsboom, G. et al. Glutamine Metabolism Regulates the Plunpotency Transcription Factor OCT4. Cell Rep. 16, 323-332 (2016).
Bae, K. N., Dai, Y., Vieweg, J. & Siemann, D. W. Hypoxia regulates SOX2 expression to promote prostate cancer cell invasion and sphere formation. Am. J. Cancer Res. 6, 1078-1088 (2016).
Sedoris, K. C., Thomas, S. D. & Miller, D. M. Hypoxia induces differential translation of enolase/MBP-1. BMC Cancer 10, (2010).
Nie, Z. et al c-Myc Is a Universal Amplifier of Expressed Genes in Lymphocytes and Embryonic Stem Cells. Cell 151, 68-79 (2012).
Wahlström, T. & Arsenian Henriksson, M. Impact of MYC in regulation of tumor cell metabolism. Biochim. Biophys. Atca—Gene Regul. Mech. 1849, 563-569 (2015).
Dang, C. V. Rethinking the warburg effect with Myc micromanaging glutamine metabolism. Cancer Res. 70, 859-862 (2010).
Elkin, S. R. et al A systematic analysis reveals heterogeneous changes in the endocytic activities of cancer cells. Cancer Res. 75, 4640-4650 (2015).
Yoshida, S., Pacitto, R., Inoki, K. & Swanson, J. Macropinocytosis, mTORC1 and cellular growth control. Cell Mol. Life Sci. 75, 1227-1239 (2018).
Mellman, I. & Yarden, Y. Endocytosis and cancer. Cold Spring Harb. Perspect. Biol. 5, a016949 (2013).
Tai, C.-Y., Mysore, S. P., Chiu, C. & Schuman, E. M. Activity-regulated N-cadherin endocytosis. Neuron 54, 771-785 (2007).
Asano, K. et al Correlation of N-cadherin expression in high grade gliomas with tissue invasion. J. Neuroncol. 70, 3-15 (2004).
Zhang, C. S. et al. The lysosomal v-ATPase-ragulator complex is a common activator for AMPK and mTORC1, acting as a switch between catabolism and anabolism. Cell Metab. 20, 526-540 (2014).
Nada, S., Mori, S., Takahashi, Y. & Okada, M. P18/LAMTOR1: A late endosome/lysosome-specific anchor protein for the mTORC1/MAPK signaling pathway. Methods in Enzymology 535, (Elsevier Inc., 2014).
Sato, T. et al Rheb protein binds CAD (carbamoyl-phosphate synthetase 2, aspartate transcarbamoylase, and dihydroorotase) protein in a GTP- And effector domain-dependent manner and influences its cellular localization and carbamoylphosphate synthetase (CPSase) activity. J. Biol. Chem. 290, 1096-1105 (2015).
Zwiewka, M., Nodzynski, T., Robert, S., Vanneste, S. & Friml, J. Osmotic Stress Modulates the Balance between Exocytosis and Clathrin-Mediated Endocytosis in Arabidopsis thaliana. Mol. Plant 8, 1175-1187 (2015).
Bricker, J. L., Chu, S. & Kempson, S. A. THE BGT1 TRANSPORTER IN MDCK CELLS. (2003).
Best, L. et al Contrasting effects of glycerol and urea transport on rat pancreatic β-cell function. Cell Physiol. Biochem. 23, 255-264 (2009).
Hönscher, C. et al Cellular metabolism regulates contact sites between vacuoles and mitochondria. Dev. Cell 30, 86-94 (2014).
El-Kadi, S. W., Baldwin, R. L., McLeod, K. R., Sunny, N. E. & Bequette, B. J. Glutamate Is the Major Anaplerotic Substrate in the Tricarboxylic Acid Cycle of Isolated Rumen Epithelial and Duodenal Mucosal Cells from Beef Cattle. J. Nutr. 139, 869-875 (2009).
Tong, X., Zhao, F. & Thompson, C. B. The molecular determinants of de novo nucleotide biosynthesis in cancer cells. Curr. Opin. Genet. Dev. 19, 32-37 (2009).
Wang, X. et al Purine synthesis promotes maintenance of brain tumor initiating cells in glioma. Nat. Neurosci. 20, 661 (2017).
Rvu, J. M., Lee, S. H., Seong, J. K. & Han, H. J. Glutamine contributes to maintenance of mouse embryonic stem cell self-renewal through PKC-dependent downregulation of HDAC1 and DNMT1/3a. Cell Cycle 14, 3292-3305 (2015).
Shin, C. S. et al. The glutamate/cystine xCT antiporter antagonizes glutamine metabolism and reduces nutrient flexibility. Nat. Commun. 8, 15074 (2017).
Metallo, C. M. et al Reductive glutamine metabolism by IDH1 mediates lipogenesis under hypoxia. Nature 481, 380 (2011).
Puskas, F., Gergely, P. J., Banki, K. & Perl, A. Stimulation of the pentose phosphate pathway and glutathione levels by dehydroascorbate, the oxidized form of vitamin C. FASEB J. Off Publ. Fed Am. Soc. Exp. Biol. 14, 1352-1361 (2000).
Cruzat, V., Rogero, M. M., Keane, K. N., Curi, R. & Newsholme, P. Glutamine: Metabolism and immune function, supplementation and clinical translation. Nutrients 10, (2018).
Sletta, K. Y. et al. Oxygen-dependent regulation of tumor growth and metastasis in human breast cancer xenografts. PLoS One 12, (2017).
Stepień, K., Ostrowski, R. P. & Matyla, E. Hyperbaric oxygen as an adjunctive therapy in treatment of malignancies, including brain tumours. Med Oncol. 33, 1-9 (2016).
Menon, S. G. & Goswami, P. C. A redox cycle within the cell cycle: Ring in the old with the new. Oncogene 26, 1101-1109 (2007).
Smith, J., Ladi, E., Mayer-Proschel, NI. & Noble, M. Redox state is a central modulator of the balance between self-renewal and differentiation in a dividing glial precursor cell. Proc. Natl. Acad. Sci. U.S.A 97, 10032-7 (2000).
Aquilano, K., Baldelli, S. & Ciriolo, M. R. Glutathione: New roles in redox signalling for an old antioxidant. Front. Pharmacol. 5 AUG, 1-12 (2014).
Jeong, E. M. et al. Real-Time Monitoring of Glutathione in Living Cells Reveals that High Glutathione Levels Are Required to Maintain Stem Cell Function. Stem Cell Reports 10, 600-614 (2018).
Circu, M. L. & Aw, T. Y. Glutathione and modulation of cell apoptosis. Biochim. Biophys. Acta-Mol. Cell Res. 1823, 1767-1777 (2012).
Polewski, M. D., Reveron-Thornton, R. F., Cherryholmes, G. A., Marinov, G. K. & Aboody, K. S. SLC7A11 Overexpression in Glioblastoma Is Associated with Increased Cancer Stem Cell-Like Properties. Stem Cells Dev. 26, 1236-1246 (2017).
Timmerman, L. A. et al Glutamine Sensitivity Analysis Identities the xCT Antiporter as a Common Triple-Negative Breast Tumor Therapeutic Target. Cancer Cell 24, 450-465 (2013).
Hasegawa, M. et al. Functional interactions of the cystine/glutamate antiporter, CD44V and MUC1-C oncoprotein in triple-negative breast cancer cells. Oncotarget 7, 11756-11769 (2016).
Liu, X. et al Cystine transporter regulation of pentose phosphate pathway dependency and disulfide stress exposes a targetable metabolic vulnerability in cancer. Nat. Cell Biol. 22, 476-486 (2020).
Mostafa, H. et al. Immune phenotypes predict survival in patients with glioblastoma multiforme. J. Hematol. Oncol. 9, 1-14 (2016).
Raggi, F. et al. Regulation of human Macrophage M1-M2 Polarization Balance by hypoxia and the Triggering receptor expressed on Myeloid cells-1. Front. Immunol. 8, 1-18 (2017).
Parodi, M. et al. Hypoxia modifies the transcriptome of human NK cells, modulates their immunoregulatory profile, and influences NK cell subset migration. Front. Immunol. 9, (2018).
Yan, Z. & Banerjee, R. Redox remodeling as an immunoregulatory strategy. Biochemistry 49, 1059-1066 (2010).
Banerjee, R. Redox outside the box: Linking extracellular redox remodeling with intracellular redox metabolism. J. Biol. Chem. 287, 4397-4402 (2012).
Yan, Z., Garg, S. K. & Banerjee, R. Regulatory T cells interfere with glutathione metabolism in dendritic cells and T cells. J. Biol. Chem. 285, 41525-41532 (2010).
Mak, T. W. et al. Glutathione Primes T Cell Metabolism for Inflammation. Immunity 46, 675-689 (2017).
Patra, K. C. & H-ay, N. The pentose phosphate pathway and cancer. Trends Biochem. Sci. 39, 347-354 (2014).
Lv, H. et al. Vitamin C preferentially kills cancer stem cells in hepatocellular carcinoma via SVCT-2. npj Precis. Oncol. 2, (2018).
Belin, S. et al. Antiproliferative effect of ascorbic acid is associated with the inhibition of genes necessary to cell cycle progression. PLoS One 4, (2009).
Castro, M. A., Beltrán, F. A., Brauchi, S. & Concha, I. I. A metabolic switch in brain: Glucose and lactate metabolism modulation by ascorbic acid. J. Neurochem. 110, 423-440 (2009).
O'Leary, B. R. et al. Pharmacological Ascorbate as an Adjuvant for Enhancing Radiation-Chemotherapy Responses in Gastric Adenocarcinoma. Radiat. Res. 189, 456-465 (2018).
Ghosh, A. K., Steele, R. & Ray, R. B. Functional Domains of c-myc Promoter Binding Protein 1 Involved in Transcriptional Repression and Cell Growth Regulation. Mol. Cell Biol. 19, 2880-2886 (1999).
Woolf, E. C. & Scheck, A. C. The ketogenic diet for the treatment of malignant glioma. J. Lipid Res. 56, 5-10 (2015).
Varshneya, K., Carico, C., Ortega, A. & Patil, C. G. The Efficacy of Ketogenic Diet and Associated Hypoglycemia as an Adjuvant Therapy for High-Grade Gliomas: A Review of the Literature. Cureus 7, 1-12 (2015).
Wang, Q. et al Ketogenesis contributes to intestinal cell differentiation. Cell Death Differ. 24, 458-468 (2017).
Nagpal, R. et al Human-origin probiotic cocktail increases short-chain fatty acid production via modulation of mice and human gut microbiome. Sci. Rep. 8, 12649 (2018).
Mackmull, Ni. T. et al Histone deacetylase inhibitors (HDACi) cause the selective depletion of bromodomain containing proteins (BCPs). Mol. Cell Proteomics 14, 1350-1360 (2015).
Delmore, J. E. et. al. BET bromodomain inhibition as a therapeutic strategy to target c-Myc. Cell 146, 904-917 (2011).
Chen, J., Zhao, K. N. & Vitetta, L. Effects of intestinal microbial-elaborated butyrate on oncogenic signaling pathways. Nutrients 11, 1-26 (2019).
Han, A., Bennett, N., Ahmed, B., Whelan, J. & Donohoe, D. R. Butyrate decreases its own oxidation in colorectal cancer cells through inhibition of histone deacetylases. Oncotarget 9, 27280-27292 (2018).
Han, A. et al Cellular Metabolism and Dose Reveal Carnitine-Dependent and -Independent Mechanisms of Butyrate Oxidation in Colorectal Cancer Cells. J. Cell. Physiol. 231, 1804-1813 (2016).
Rada-Iglesias, A. et al. Butyrate mediates decrease of histone acetylation centered on transcription start sites and down-regulation of associated genes. Genome Res. 17, 708-719 (2007).
Chaput, N. et al. Baseline gut microbiota predicts clinical response and colitis in metastatic melanoma patients treated with ipilimumab. Ann. Oncol. 28, 1368-1379 (2017).
Gopalakrishnan, V. et al. Gut microbiome modulates response to anti-PD-1 immunotherapy in melanoma patients. Science (80-. ). 359, 97-103 (2018).
Pfirschke, C. et al. Immunogenic Chemotherapy Sensitizes Tumors to Checkpoint Blockade Therapy. Immunity 44, 343-354 (2016).
Wang, Q. et al. Ketogenesis contributes to intestinal cell differentiation. Cell Death Differ. 24, 458-468 (2017).
Johnson, R. C. & Kirabo Jackson, C. Reducing inequality through dynamic complementarity: Evidence from head start and public school spending. Am. EcOn. J. Econ. Policy 11, 310-349 (2019).

The disclosure above encompasses multiple distinct inventions with independent utility. While each of these inventions has been disclosed in a particular form, the specific embodiments disclosed and illustrated above are not to be considered in a limiting sense as numerous variations are possible. The subject matter of the inventions includes all novel and non-obvious combinations and subcombinations of the various elements, features, functions and/or properties disclosed above and inherent to those skilled in the art pertaining to such inventions. Where the disclosure or subsequently filed claims recite “a” element, “a first” element, or any such equivalent term, the disclosure or claims should be understood to incorporate one or more such elements, neither requiring nor excluding two or more such elements.

Applicant(s) reserves the right to submit claims directed to combinations and subcombinations of the disclosed inventions that are believed to be novel and non-obvious. Inventions embodied in other combinations and subcombinations of features, functions, elements and/or properties may be claimed through amendment of those claims or presentation of new claims in the present application or in a related application. Such amended or new claims, whether they are directed to the same invention or a different invention and whether they are different, broader, narrower or equal in scope to the original claims, are to be considered within the subject matter of the inventions described herein.

1. A method for treating cancer comprising:
adjusting the concentration of selected metabolites in a blood supply to yield a perfusate with metabolite concentrations that promote terminal cellular differentiation in a tumor; and
delivering the perfusate to an artery of a patient.

2. The method of claim 1, wherein the concentration of selected metabolites in a blood supply are adjusted to reduce the osmotic pressure of the perfusate.

3. The method of claim 2, wherein adjusting the concentration of selected metabolites in a blood supply includes adjusting the concentration of glycerol to reduce the osmotic pressure of the perfusate.

4. The method of claim 4, wherein adjusting the concentration of selected metabolites in a blood supply includes increasing the concentration of glycerol in the perfusate.

5. The method of claim 1, wherein adjusting the concentration of selected metabolites in a blood supply includes maintaining a selected redox potential of the perfusate.

6. The method of claim 5, wherein the selected redox potential corresponds to a selected concentration of intra-cellular glutathione that promotes apoptosis in a tumor.

7. The method of claim 6, wherein the selected redox potential of the perfusate is maintained by adjusting the concentration of ascorbic acid in the perfusate.

8. The method of claim 1, wherein adjusting the concentration of selected metabolites in a blood supply includes adjusting the concentration of glutamine in the perfusate to downregulate histone deacetylases.

9. The method of claim 8, wherein adjusting the concentration of selected metabolites in a blood supply includes reducing the concentration of glutamine in the perfusate.

10. The method of claim 9, wherein adjusting the concentration of selected metabolites in a blood supply includes reducing the concentration of glutamine in the perfusate to a concentration selected to reduce cysteine exporting from a tumor cell.

11. The method of claim 1, wherein adjusting the concentration of selected metabolites in a blood supply includes adjusting the concentration of selected metabolites in the perfusate to signal starvation.

12. The method of claim 11, wherein adjusting the concentration of selected metabolites in a blood supply includes adjusting the concentration of butyrate in the perfusate to signal starvation.

13. The method of claim 12, wherein adjusting the concentration of butyrate in the perfusate to signal starvation includes increasing the concentration of butyrate in the perfusate.

14. The method of claim 11, wherein adjusting the concentration of selected metabolites in a blood supply includes adjusting the concentration of ketone bodies in the perfusate to signal starvation.

15. The method of claim 14, wherein adjusting the concentration of ketone bodies in the perfusate to signal starvation includes increasing the concentration of ketone bodies in the perfusate.

16. The method of claim 1, wherein the concentration of selected metabolites in a blood supply are adjusted to concentrations selected to regulate expression of the Yamanaka stem cell factors in the perfusate.

17. The method of claim 1, wherein the concentration of selected metabolites in a blood supply are adjusted to concentrations selected to restore a patient's compromised immune response to a tumor to a baseline immune response.

18. The method of claim 17, wherein the concentration of selected metabolites in a blood supply are adjusted to concentrations selected to restore extracellular oxidation reduction potential in an environment proximate a tumor to within a baseline range of extracellular oxidation reduction potential.

19. A method for treating cancer comprising:
adjusting the concentration of glycerol in a blood supply to yield a perfusate with reduced osmotic pressure;
adjusting the concentration of ascorbic acid in the perfusate to maintain a selected redox potential;
adjusting the concentration of glutamine in the perfusate to downregulate histone deacetylases;
adjusting the concentration of butyrate in the perfusate to signal starvation; and
delivering the perfusate to an artery of a patient;
wherein the concentrations of glycerol, ascorbic acid, glutamine, and butyrate are selected to promote terminal cellular differentiation in a tumor.

20. The method of claim 19, wherein:
adjusting the concentration of glycerol in a blood supply to yield a perfusate with reduced osmotic pressure includes increasing the concentration of glycerol;
adjusting the concentration of ascorbic acid in the perfusate to maintain a selected redox potential includes increasing the concentration of ascorbic acid;
adjusting the concentration of glutamine in the perfusate to downregulate histone deacetylases includes decreasing the concentration of glutamine; and
adjusting the concentration of butyrate in the perfusate to signal starvation includes increasing the concentration of butyrate.
